(19) United States
(12) Patent Application Publication
(10) Pub. No.: US 2023/0157641 A1
(43) Pub. Date: May 25, 2023

(54) ABSOLUTE ISOLATED LIGHT PATH STRUCTURE, AND SEAT RING AND TOILET DEAT USING ASBOLUTE ISOLATED LIGHT PATH STRUCTURE

(71) Applicants: Guangzhou Eaphonetech Co., Ltd., Guangdong (CN); Guangzhou Xinxiangsui Technology Co., Ltd., Guangdong (CN)

(72) Inventors: Pei Wang, Guangdong (CN); Jinhe Luo, Guangdong (CN)

(21) Appl. No.: 17/920,363

(22) PCT Filed: Mar. 30, 2021
(86) PCT No.: PCT/CN2021/083997

§ 371 Date: Oct. 20, 2022

(30) Foreign Application Priority Data
Apr. 20, 2020 (CN) 202010315524.5

(51) Int. Cl. A61B 5/00; A47K 13/24; A61B 5/282; A61B 5/26; A61B 5/0205

(52) U.S. Cl. CPC A61B 5/6891; A47K 13/24; A61B 5/282; A61B 5/26; A61B 5/0205; A61B 5/02427

(57) ABSTRACT

Disclosed are absolute isolated light path structure, and a seat ring and a toilet seat using the absolute isolated light path structure. The absolute isolated light path structure comprises a light blocking plate, and a light emitting device and a light receiving device separated by the light blocking plate, the light blocking plate is configured for blocking light propagation between the light emitting device and the light receiving device, thereby avoiding the interference from the light emitted by the light emitting device to the light receiving device.

### TECHNICAL FIELD

The present disclosure relates to the field of toilet seat technologies, and specifically to an absolute isolated light path structure, and a seat ring and a toilet seat having the same.

### BACKGROUND

A closestool, also known as a toilet, is a necessary fixture in people's daily life, and smart toilet seats are popular because of its medical and health effects. At present, a smart toilet seat is able to collect human biometric characteristics, such as electrocardiographic (ECG) signals, to achieve the purpose of monitoring health status of human bodies.

Photo-plethysmography (PPG) is a technique for acquiring plethysmogram of organs optically, which is usually implemented by irradiating the skin with a pulse oximeter and measuring the amount of change in light absorption. The PPG signal may be adopted to calculate the body's oxyhemoglobin saturation, heart rate, blood pressure and other physiological indicators. The PPG signal is generally acquired via optical signals with two wavelengths of 940 nm and 650 nm, and by ways of transmission absorption (e.g., at the fingertips, earlobes) or reflection (e.g., on the forehead).

According to the existing optical technology for acquiring the PPG signals by reflection, the light-emitting and light-receiving portions may share the same piece of light-transmitting material for implementing the light-guiding and waterproofing processing no matter in discrete devices or an integrated sensor, as shown in FIG. 1. Both the light-emitting light source and the light-receiving photocell contact the human body across the same light-transmitting medium, and no light-transmitting material enables the light energy transmission of 100%, which may inevitably cause total reflection, refraction or other phenomena. Thus, the light-receiving portion may inevitably suffers from partial direct radiation of the light source signal, which appears as spurs parasitic on the signal waveform when the PPG signal is weak and thereby deteriorates the signal quality.

### SUMMARY

In view of this, it is necessary to provide an absolute isolated light path structure to avoid interference from the light emitting portion to the light receiving portion, and also a seat ring and toilet seat having the absolute isolated light path structure.

Provided is an absolute isolated light path structure, which includes: a light blocking plate; and a light emitting device and a light receiving device separated by the light blocking plate, wherein the light blocking plate is configured to block light propagation between the light emitting device and the light receiving device.

Furthermore, the light emitting device includes a light emitter and an emitter light-transmitting sheet disposed above the light emitter; the light receiving device includes a light receiver and a receiver light-transmitting sheet disposed above the light receiver; and the light blocking plate is disposed between the light emitter and the light receiver and extends between the emitter light-transmitting sheet and the receiver light-transmitting sheet.

Furthermore, the emitter light-transmitting sheet and the receiver light-transmitting sheet have respective upper surfaces flush with an upper surface of the light blocking plate.

In addition, provided is a seat ring having an absolute isolated light path structure, which includes: a body signal extraction unit, wherein the body signal extraction unit is fitted with the absolute isolated light path structure described above and configured to extract a PPG signal from a human body.

Furthermore, the seat ring further includes a seat surface and a bottom surface opposite to the seat surface, wherein the seat surface is provided with a mounting hole penetrating therethrough; and

the light emitting device and the light receiving device are mounted in, with a gap around the light emitting device and the light receiving device in the mounting hole being filled with a light blocking material that forms the light blocking plate.

Furthermore, the mounting hole is entirely filled with the light blocking material provided with two through holes; and

the two through holes include a light emitter mounting hole and a light receiver mounting hole configured to mount the light emitting device and the light receiving device, respectively.

Furthermore, the light emitter mounting hole and the light receiver mounting hole are provided in parallel.

Furthermore, the emitter light-transmitting sheet, the receiver light-transmitting sheet, and the light blocking plate have respective upper surfaces flush with the seat surface of the seat ring.

In addition, provided is a toilet seat, which includes the absolute isolated light path structure described above and a weak electrical signal acquisition circuit for acquiring an ECG signal of a human body.

The weak electrical signal acquisition circuit includes two signal acquisition units that include two signal receiving electrode sheets and two operational amplifiers, wherein the two operational amplifiers form a differential circuit; and

the differential circuit further includes a first feedback resistor R3, a second feedback resistor R3′ and a negative feedback circuit provided between the first feedback resistor R3 and the second feedback resistor R3′.

Furthermore, the two signal receiving electrode sheets are active electrode sheets, and are configured to receive common mode signals and differential mode signals;

the negative feedback circuit is formed by a single resistor or a network assembly consisting of a plurality of electronic components and having a frequency characteristic; or,

the negative feedback circuit includes a network assembly having a frequency characteristic of all pass, low pass, high pass, band pass or band resistance.

The present disclosure has following advantageous technical effects.

1. According to the present disclosure, the light blocking plate is provided between the light emitting device and the light receiving device, such that the light emitting device and the light receiving device can emit and receive light independently, thereby preventing the light emitted by the light emitting device from interfering with the light receiving device.

2. The light path of the light emitting device is completely isolated from that of the light receiving device, such that the light emitted by the light emitting device is prevented from interfering with the light receiving device before being reflected by the human body, thereby improving the quality of the PPG signal acquired from thick fatty parts.

3. By taking the two signal acquisition units as pre-input electrodes of an amplification circuit and adopting the in-phase differential input mode, the amplitude ratio of differential mode signals to common mode signals as sent to a post-operational amplifier is increased, and the purpose of improving the signal-to-noise ratio of the input signal can be achieved, such that the toilet seat can extract ECG signals from the human body effectively.

4. When people use the toilet, the toilet seat fitted with the weak signal acquisition circuit can collect ECG signals from legs, and can still effectively extract ECG signals from human body even in dry winters.

### BRIEF DESCRIPTION OF THE DRAWINGS

FIG. 1 is a schematic diagram of a light path structure of a toilet seat in the prior art;

FIG. 2 is a schematic diagram of a light path structure of an absolute isolated light path structure according to an embodiment of the present disclosure;

FIG. 3 is an enlarged schematic structural diagram of a part of a mounting hole of a body signal extraction unit of a seat ring having an absolute isolated optical path structure according to an embodiment of the present disclosure;

FIG. 4 is a schematic structural diagram of the seat ring having an absolute isolated optical path structure according to an embodiment of the present disclosure; and

FIG. 5 is a schematic structural diagram of a weak signal acquisition circuit of the toilet seat according to an embodiment of the present disclosure.

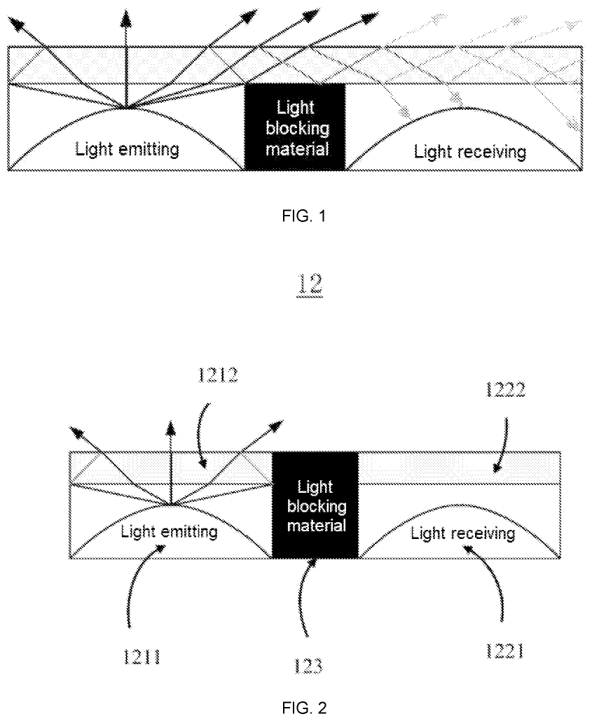

### DETAILED DESCRIPTION

The present disclosure will be further illustrated in detail in combination with the specific embodiments and accompanying drawings.

### EXAMPLE 1

Referring to FIG. 2, it illustrates an absolute isolated light path structure, which includes a light blocking plate 123, and a light emitting device 121 and a light receiving device 122 separated by the light blocking plate 123. The light blocking plate 123 is configured to block light propagation between the light emitting device 121 and the light receiving device 122.

Furthermore, the light emitting device 121 includes a light emitter 1211 and an emitter light-transmitting sheet 1212 disposed above the light emitter 1211; the light receiving device 122 includes a light receiver 1221 and a receiver light-transmitting sheet 1222 disposed above the light receiver 1221; and the light blocking plate 123 is disposed between the light emitter 1211 and the light receiver 1221 and extends between the emitter light-transmitting sheet 1212 and the receiver light-transmitting sheet 1222.

Furthermore, the emitter light-transmitting sheet 1212 and the receiver light-transmitting sheet 1222 have respective upper surfaces flush with an upper surface of the light blocking plate 123.

Specifically, the light blocking plate 123 completely isolates the light transmitting device 121 from the light receiving device 122, such that the light emitted from the light emitter 1211 cannot transmit to the light receiver 1221 via the shared light guiding plate, thereby reducing the light interference signals as received by the light receiver 1221.

Preferably, the light blocking material is made of a metallic material or other light-opaque material.

In this embodiment, the light blocking plate 123 completely isolates the light transmitting device 121 from the light receiving device 122, thereby preventing the light emitted by the light emitting device 121 from interfering the light receiving device 122.

### EXAMPLE 2

Referring to FIG. 4, the present disclosure further discloses a seat ring 10 having an absolute isolated light path structure. As shown in FIG. 4, the seat ring 10 includes a body signal extraction unit, and the body signal extraction unit is fitted with the absolute isolated light path structure described above and configured to extract a PPG signal from a human body.

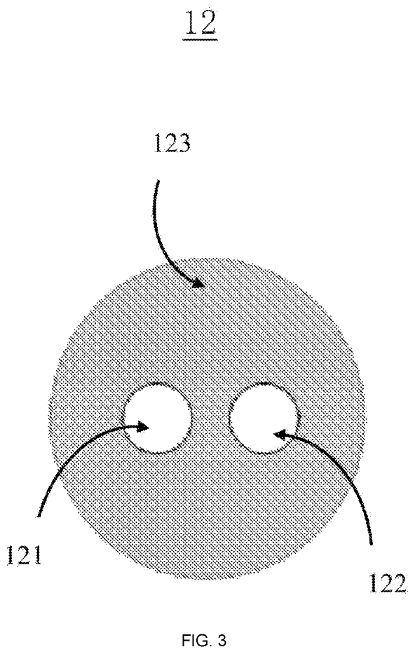

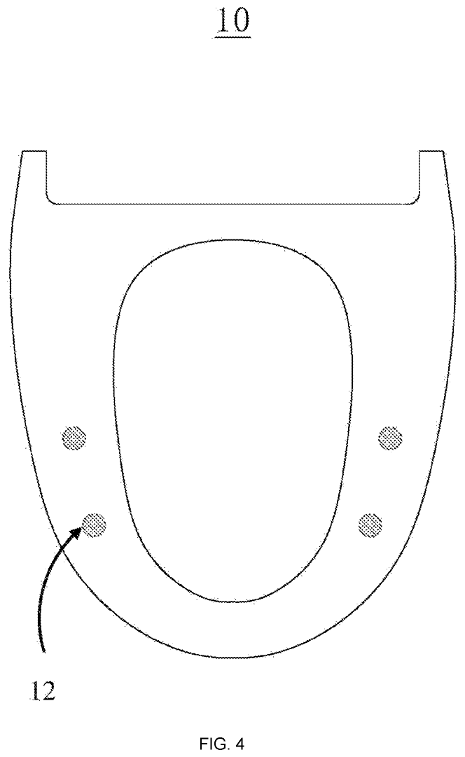

Furthermore, referring to FIG. 3, the seat ring 10 has a seat surface and a bottom surface opposite to the seat surface, and the seat surface is provided with a mounting hole 12 penetrating therethrough. The light emitting device 121 and the light receiving device 122 are mounted in the mounting hole 12, with a gap around the light emitting device 121 and the light receiving device 122 in the mounting hole 12 being filled with a light blocking material that forms the light blocking plate 123. The mounting hole 12 is entirely filled with the light blocking material provided with two through holes. The two through holes include a mounting hole for the light emitter 1211 and a mounting hole for the light receiver 1221, which are configured to mount the light emitting device 121 and the light receiving device 122 respectively. In addition, the mounting hole for the light emitter 1211 and the mounting hole for the light receiver 1221 are provided in parallel.

Furthermore, the emitter light-transmitting sheet 1212, the receiver light-transmitting sheet 1222, and the light blocking plate 123 have respective upper surfaces flush with the seat surface of the seat ring 10.

Specifically, the seat ring 10 having the absolute isolated light path structure according to this embodiment may be mounted either on a normal toilet seat without heating and flushing functions or on a smart toilet seat with heating and flushing functions and functions of detecting human physiological parameters (e.g. body fat parameters, weight parameters, ECG signals, etc.).

According to this embodiment, in the toilet seat fitted with the absolute isolated light path structure, the light path of the light emitting device 121 is completely isolated from that of the light receiving device 122, such that the light emitted by the light emitting device 121 is prevented from interfering the light receiving device 122 before being reflected by the human body, thereby improving the quality of the PPG signals as acquired from thick fatty parts.

### EXAMPLE 3

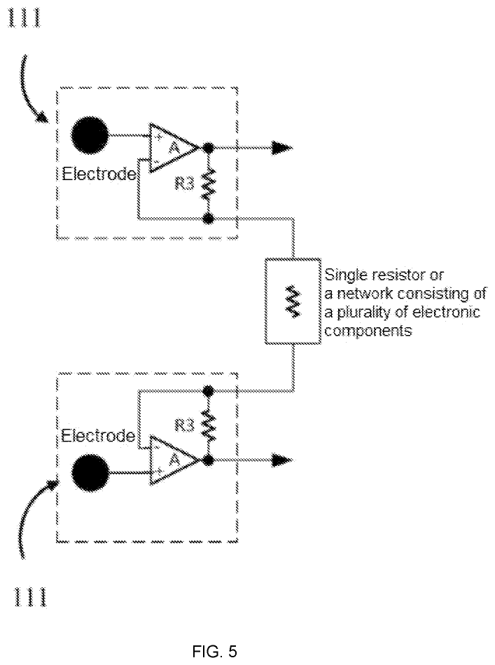

The present disclosure further discloses a toilet seat that includes the absolute isolated light path structure described above and a weak electrical signal acquisition circuit for acquiring an ECG signal of a human body. Referring to FIG. 5, the weak electrical signal acquisition circuit includes two signal acquisition units that include two signal receiving electrode sheets 111 and two operational amplifiers. The two operational amplifiers form a differential circuit, and the differential circuit further includes a first feedback resistor R3, a second feedback resistor R3′ and a negative feedback circuit provided between the first feedback resistor R3 and the second feedback resistor R3′.

Furthermore, the two signal receiving electrode sheets 111 are active electrode sheets, and are configured to receive common mode signals and differential mode signals. The negative feedback circuit is formed by a single resistor or a network assembly consisting of a plurality of electronic components and having a frequency characteristic; or, the negative feedback circuit includes a network assembly having a frequency characteristic of all pass, low pass, high pass, band pass or band resistance.

Specifically, the toilet seat according to this embodiment may include both an ordinary toilet seat without heating and flushing functions and a smart toilet seat with the heating and flushing functions.

Specifically, when people use the toilet, the toilet seat fitted with the weak signal acquisition circuit can collect ECG signals from legs, and can still effectively extract ECG signals of human body even in dry winters.

According to this embodiment, the weak signal acquisition circuit can reduce the interference of external electrical signals to the signal acquisition unit and improves the accuracy of the system in acquiring ECG signals of the human body.

It shall be noted that the above description is only the preferred embodiments of the present disclosure, and is not intended to limit the present disclosure. For those skilled in the art, various modifications and changes can be made to the present disclosure. Any modification, equivalent replacement, improvement, and the like as made according to the spirit and principle of the present disclosure shall be regarded as within the protection scope of the present disclosure.

1. An absolute isolated light path structure, comprising:
a light blocking plate; and
a light emitting device and a light receiving device separated by the light blocking plate,
wherein the light blocking plate is configured to block light propagation between the light emitting device and the light receiving device.

2. The absolute isolated light path structure of claim 1, wherein the light emitting device comprises a light emitter and an emitter light-transmitting sheet disposed above the light emitter;
the light receiving device comprises a light receiver and a receiver light-transmitting sheet disposed above the light receiver; and
the light blocking plate is disposed between the light emitter and the light receiver and extends between the emitter light-transmitting sheet and the receiver light-transmitting sheet.

3. The absolute isolated light path structure of claim 2, wherein the emitter light-transmitting sheet and the receiver light-transmitting sheet have respective upper surfaces flush with an upper surface of the light blocking plate.

4. A seat ring having an absolute isolated light path structure, comprising a body signal extraction unit, wherein the body signal extraction unit is fitted with the absolute isolated light path structure of claim 1 and configured to extract a PPG signal from a human body.

5. The seat ring of claim 4, wherein the seat ring has a seat surface and a bottom surface opposite to the seat surface, and the seat surface is provided with a mounting hole penetrating therethrough; and
the light emitting device and the light receiving device are mounted in the mounting hole, with a gap around the light emitting device and the light receiving device in the mounting hole being filled with a light blocking material that forms the light blocking plate.

6. The seat ring of claim 5, wherein the mounting hole is entirely filled with the light blocking material provided with two through holes; and
the two through holes comprise a light emitter mounting hole and a light receiver mounting hole configured to mount the light emitting device and the light receiving device, respectively.

7. The seat ring of claim 6, wherein the light emitter mounting hole and the light receiver mounting hole are provided in parallel.

8. The seat ring of claim 5, wherein the emitter light-transmitting sheet, the receiver light-transmitting sheet, and the light blocking plate have respective upper surfaces flush with the seat surface of the seat ring.

9. A toilet seat, comprising the absolute isolated light path structure of claim 1 and a weak electrical signal acquisition circuit for acquiring an electrocardiographic signal of a human body, wherein
the weak electrical signal acquisition circuit comprises two signal acquisition units that comprise two signal receiving electrode sheets and two operational amplifiers, wherein the two operational amplifiers form a differential circuit; and
the differential circuit further comprises a first feedback resistor R3, a second feedback resistor R3′ and a negative feedback circuit provided between the first feedback resistor R3 and the second feedback resistor R3′.

10. The toilet seat of claim 9, wherein the two signal receiving electrode sheets are active electrode sheets, and are configured to receive common mode signals and differential mode signals;
the negative feedback circuit is formed by a single resistor or a network assembly consisting of a plurality of electronic components and having a frequency characteristic; or,
the negative feedback circuit comprises a network assembly having a frequency characteristic of all pass, low pass, high pass, band pass or band resistance.
